(19) United States
(12) Patent Application Publication
(10) Pub. No.: US 2008/0282602 A1
(43) Pub. Date: Nov. 20, 2008

(54) Therapeutic Skin Treatment Composition and Method

(76) Inventor: Sharon K. Yingst, Celina, OH (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 12/182,608

(22) Filed: Jul. 30, 2008

Related U.S. Application Data
(63) Continuation-in-part of application No. 11/279,341, filed on Apr. 11, 2006, now abandoned.
Application No. 11/279,341 is a division of application No. 11/063,432, filed on Feb. 22, 2005, now abandoned.

(51) Int. Cl. A61F 7/00; C11C 5/02; F23D 3/16

(52) U.S. Cl. 44/275; 431/289; 607/96

(57) ABSTRACT

A method of using a novel candle apparatus at least includes: candle housing; candle fuel deposited in the candle housing, the candle fuel at least including non-toxic solidified therapeutic skin substance; and a candle fuel liquefier; wherein the candle fuel is adapted to, upon application of the fuel liquefier, liquefy into a therapeutic substance adapted for human skin. In one refinement, the therapeutic skin substance is massage oil. In another refinement, the therapeutic skin substance is a soap agent for cleansing skin, and the like. A therapeutic skin treatment method at least includes: a) providing a candle that at least includes, candle housing, candle fuel deposited in the candle housing, the candle fuel at least including non-toxic solidified therapeutic oil, and a candle fuel liquefier; b) liquefying the candle fuel into therapeutic oil for human skin; and c) applying to the skin, therapeutic oil produced in element b).

### CROSS-REFERENCE TO RELATED APPLICATIONS

This application is a Continuation-in-Part of U.S. patent application Ser. No. 11/279,341, filed Apr. 11, 2006, for “Therapeutic Skin Treatment Method, which is a divisional of application Ser. No. 11/063,432 entitled “CANDLE USING SOLIDIFIED SKIN CARE AGENTS FOR FUEL, AND PROVIDING SKIN CARE LIQUID AS AN OPERATIONAL END PRODUCT” filed on 22 Feb. 2005, incorporates the contents thereof, and claims priority thereto. This application further claims priority to and hereby incorporates by reference U.S. Provisional Patent Application No. 60/578,064 entitled “Massage Oil Candle” and filed on 9 Jun. 2004.

### STATEMENT REGARDING FEDERALLY SPONSORED RESEARCH

Not applicable.

### BACKGROUND OF THE INVENTION

The present invention generally relates to products useful for providing therapeutic massage and for methods of therapeutic skin treatment, specifically including using melted skin care products.

Candles are often used to create an environment conducive to calm and relaxation by combining visual effects, colors and aromas. It is common for people to receive therapeutic skin treatment—either self-administered, or administered by others—with burning candles in the vicinity. Such therapeutic skin treatments may include hand and body massages with various massage oils, and bathing or showering with therapeutic body washes and soaps.

A variety of candles are known to those skilled in the art, and a review of said art demonstrates that there are is a nearly infinite variety of compositions that can be used to create a candle, so long as the candle (or lamp) provides a liquefiable, combustible fuel source. A variety of books and web archives list the may potential ingredients that might be include in a candle. What is desirable, but unaddressed by the prior art, is providing simple candle products which directly combine the traditional attributes and benefits of candles with therapeutic skin care agents in simple articles of manufacture. For example, U.S. Pat. No. 6,730,137 discloses candles incorporating hydrogenated vegetable oil, which candles are gentle to the touch, and can to some extent, be rubbed into the hands (and on the skin).

Ohtuski, et al., Japan Patent No. 63,214,255A is exemplary of a variety of paraffin wax compositions that may be melted and “dropped on the surface of human skin” without causing severe burns. Ohtuski and the like art does not, however, teach that lowering the melting point of the disclosed wax is a desired property, and it is a property of paraffin based compositions that direct exposure to human skin may be injurious, painful, or at least uncomfortable. Thus, Ohtuski simply uses existing paraffin candles to drop scalding wax on the skin. Ohtuski does not, however teach that lowering the melting point of the disclosed wax is a desired property, let alone a most desirable property. In the spa environment, a relaxing atmosphere is desired, and application of a warm composition that is soothing to the patient is desired. Application of melted paraffin wax to the skin of patients is known to be uncomfortable, and the solidification of that wax to remove hair or to be effective as a peel is a desired quality.

Brown, U.S. Pat. No. 4,360,387, demonstrates some of the variety of candle fuels that are known, including the inclusion of jojoba oil. Brown, teaches that when less than 25% jojoba oil is used, a grainy, low strength, slushy appearance occurs in the candle wax, such that compositions containing less than 25% jojoba oil are undesirable. Brown thus teaches away from utilizing jojoba oil at low percentages.

What is not taught or suggested by the prior art are methods of using candles which directly include massage oil or other non-toxic therapeutic skin care agents as candle fuel, and in which the candle fuel is liquefied during normal candle operation to produce usable therapeutic skin care agents, such as massage oil. It is also desirable to provide candle products with the above attributes, in which the candle fuel mixture can be liquefied either by burning a conventional wick, or in a “wickless” manner with a simple heat source.

### BRIEF SUMMARY OF THE INVENTION

The invention is embodied, at least in part, by a candle providing a liquefiable candle fuel comprising a non-toxic solidified therapeutic oil adapted to be applied in its liquid state as a therapeutic substance adapted for use on human skin. A preferred embodiments is a liquefiable massage oil composition, comprising a massage oil product that is liquid when warmed to just above body temperature, and is usable only when it is warmed. Heretofore, there has been no other products possessing these features.

Accordingly, the present invention has been developed to provide a candle apparatus at least including: candle housing; candle fuel deposited in the candle housing, the candle fuel at least including non-toxic solidified therapeutic skin substance; and a candle fuel liquefier; wherein the candle fuel is adapted to, upon application of the fuel liquefier, liquefy into a therapeutic substance adapted for human skin.

In one refinement of the present invention, the therapeutic skin substance is massage oil. In another refinement of the present invention, the therapeutic skin substance is a soap agent for cleansing skin, and the like. The present invention has also been developed to provide a therapeutic skin treatment method at least including: a) providing a candle that at least includes, candle housing, candle fuel deposited in the candle housing, the candle fuel at least including non-toxic solidified therapeutic oil, and a candle fuel liquefier; b) liquefying the candle fuel into therapeutic oil for human skin; and c) applying to the skin, therapeutic oil produced in element b). This method is embodied in a method of therapeutically treating skin comprising providing a candle comprising: 1) a candle housing; 2) a candle body deposited in said candle housing, said candle body comprising a solidified liquefiable homogenous composition including a flammable candle fuel primarily derived from soy that burns evenly without appreciable production of soot or discoloration, two or more emollients suitable for application to skin, said emollients comprising shea butter, jojoba oil, lanolin, and aloe extracts and 3) a candle liquefier comprising a wick or other heating means; allowing liquefying of said candle body by igniting the candle liquifier for instance to provide liquefied therapeutic oil for human skin, said liquefied therapeutic oil being tolerable to the touch, said liquefied therapeutic oil remaining liquid at temperatures greater than about 35° C. and applying to the skin, liquefied therapeutic oil produced.

Another embodiment is a candle apparatus comprising a) a candle housing; b) a candle body deposited in said candle housing, said candle body comprising a solidified liquefiable homogenous composition including a flammable candle fuel primarily derived from soy that burns evenly without appreciable production of soot or discoloration, two or more emollients suitable for application to skin, said emollients comprising shea butter, jojoba oil, lanolin, and aloe extracts; and c) a candle liquefier; wherein liquefying said candle body using the candle liquifier into liquefied therapeutic oil for human skin, said liquefied therapeutic oil being tolerable to the touch, said liquefied therapeutic oil remaining liquid at temperatures greater than about 35° C.

Said candle apparatus is further embodied, wherein said candle fuel comprises from about 93 to about 98 weight percent soy flakes; from about 0.005 to about 0.7 weight percent shea butter; and from about 1 to about 6 weight percent jojoba oil. Alternatively, the candle fuel comprises from about 96 to about 98 weight percent soy flakes, from about 0.005 to about 0.7 weight percent shea butter, and from about 1 to about 2 weight percent jojoba oil. Further alternatively, the candle fuel comprises about 98 weight percent Soy 402 flakes, about 0.05 weight percent shea butter, and about 2 weight percent jojoba oil.

The present invention has been further developed to provide a solid soap article at least including: a first layer comprising soy flakes and at least one carrier oil; and a second layer comprising soy flakes, at least one carrier oil, and glycerin.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that arc illustrated in the appended drawings.

FIG. 1 is an isometric view of a first embodiment of the present-inventive candle apparatus;

FIG. 2 is an isometric view of a second embodiment of the present-inventive candle apparatus;

FIG. 3 is an isometric view of a third embodiment of the present-inventive candle apparatus;

FIG. 4 is a top view of a fourth embodiment of the present-inventive candle apparatus;

FIG. 5 is a side view of a soap article according to the present invention; and

FIG. 6 is a side view of the soap article of FIG. 5.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiments” “an embodiment,” and similar language throughout this specification may, but do not necessarily, all refer to the same embodiment.

In reviewing the known art it is apparent that none of this art teaches either the desirability for or a strategy for providing a composition wherein a liquefiable massage oil composition is provided, comprising a massage oil product that is liquid when warmed to just above body temperature, and is usable only when it is warmed. Yet it is the particular chemical composition as claimed by the Applicant that provides for a liquefiable massage oil composition that is liquid when warmed to just above body temperature. The presently disclosed composition was discovered only after engaging in extensive experimentation by which to reach the desired result of a castable candle material that maintained homogeneity when melted, cast into a candle form and then cooled into a solid, and that when liquefied, either using a wick or other heating means, provides a massage oil composition that may be applied directly to the skin, and is useful for massaging the skin. The present disclosure is the first suggestion to create a liquefiable massage oil composition that may be applied directly to the skin as the desired result. Prior to the invention disclosed herein, not only was there no teaching to direct an artisan to reach the desired result, there was no teaching that suggested even attempting to reach the composition according to the inventions.

The particular chemical composition as disclosed provides enablement for the concept of a liquefiable massage oil composition that is liquid when warmed to just above body temperature. Among the new features of the claimed method is the provision for providing a massage oil composition that when melted is tolerable to the touch and may be applied directly to the skin.

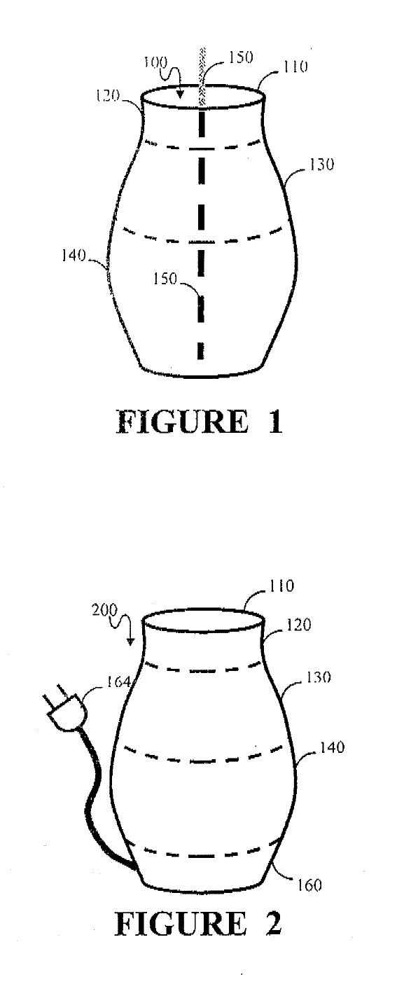

FIG. 1 illustrates a candle apparatus 100 according to a first embodiment of the present invention. A candle housing or candle container 110 contains candle fuel in the region 140. The candle housing 110 also has a region 130 for collecting liquefied massage oil (as will be described infra.), and a region 120 at the top of the housing. In the embodiment of FIG. 1, a wick 150 is used to burn and liquefy the candle fuel. The housing can be constructed of glass, ceramics, and other suitable materials, as will be appreciated by those skilled in the art.

The present inventions present new uses for solidified massage oil in the candle fuel mixture, and such uses are not typical of paraffin and petroleum waxes or oils. Toward that end, the candle fuel mixture contains soy oil substantially free of other vegetable oils such as cottonseed oil or hydrogenated vegetable oils. The candle fuel mixture further contains a carrier oil, which provides moisturizing and lubricating properties to liquefied oil when the candle fuel is melted. Exemplary carrier oils are jojoba oil, shea butter, and dehydrated aloe.

In operation, the candle wick 150 is ignited to burn the solid candle fuel mixture, which fuel is converted in the process to liquefied massage oil. A candle user may then use the massage oil as desired immediately after liquefication, of at a later time. The temperature of the massage oil—except immediately at the burning focal point of the wick—is designed to be tolerable to the touch.

The massage oil candle of the present invention can be constructed in a “wickless” version 200 as well. (See FIG. 4) That is, the candle fuel liquefier takes the form of a heating source 160 rather than an ignited wick. Activation of the heating source 160 causes the candle fuel to melt and produce the aforementioned massage oil. The heating source 160 can take on many forms according to the present invention. For example, the heating source 160 can be a powered (via an electric outlet and cord 164) heating element using an ohmic heating process. Other beating sources include candle warmers and melting pots, to name a few.

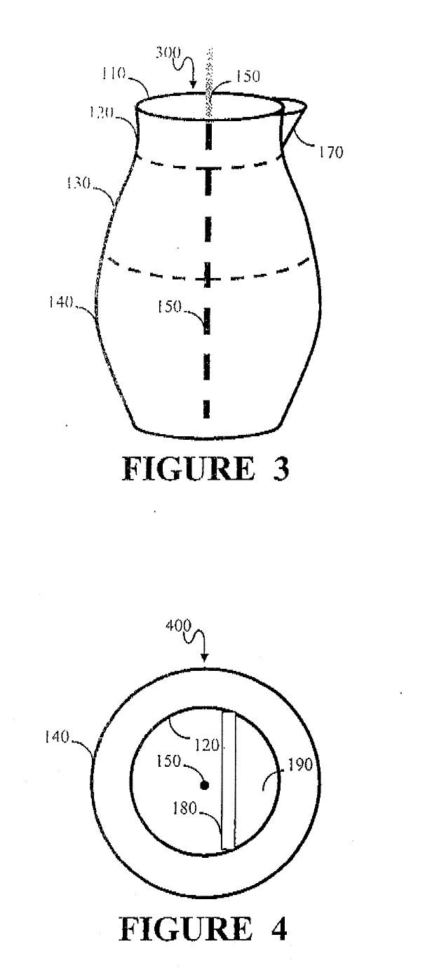

Those skilled in the art will appreciate that many variations of the candle housing are possible, given the teachings of the present invention. For example, a version 300 (see FIG. 3) of a candle apparatus contains a pouring spout 170 to facilitate pouring of the massage oil. Also, many variations of the candle housing can be constructed to provide convenient reservoirs for storing liquefied massage oil. While a simple version allows the liquefied massage oil to collect on top of the solid candle fuel mixture, other possibilities including partitioning the candle housing to allow liquefied massage oil to collect in a separate compartment from the solid candle fuel. In the version 400 of FIG. 4, a partition 180 separates the unmelted candle fuel from liquefied massage oil which collects in a reservoir 190.

Candles have been utilized for many years to provide soft lighting, derived from the burning of waxes and or oils and for the soothing effects of flickering flames produced by wicks that burn waxes or oils. Candles and or lamp fuels have utilized a wide variety of liquefiable flammable substances, including, for instance, beeswax, whale oil, kerosene, paraffin, animal fats and oils, and vegetable fats and oils. a number of these fuels produce objectionable byproducts, including soot and odors. One advantage of vegetable oil based candle fuels is the relatively complete combustion that can be achieved, thus avoiding objectionable by products such as soot and odors. Moreover, vegetable oil candle fuels, such as soybean (soy) oil candle fuels can be utilized to deliver fragrances and other useful substances that can be utilized in a variety of therapeutic methods, including massage therapy. Whereas, paraffin candles produce soot, along with potentially carcinogenic and or toxic combustion byproducts, and are derived from fossil fuel sources, soy candle fuels are clean burning, and do not produce an objectionable odor resulting from incomplete combustion, particularly from a smoldering candle wick. Thus, when paraffin based candles are burned in confined spaces, such as beauty salons and or massage parlors, a variety of known carcinogens are released and potentially concentrated in a confined space.

Soy based candle fuels are advantageous to a number of users because of their vegetable, renewable origin, and because of the ease at which the melted candle fuels may be cleaned up with traditional cleaning products. While spilled waxes from paraffin candles generally causes stains and is difficult to clean, soy candle fuel may be cleaned up with soap and water. While prior to the present disclosure, it had been known that soy candle fuels could be utilized for producing candles, the present disclosure teaches that the soy candle fuel can be used to as a basis to deliver a variety of beneficial substances, particularly, substances and compositions useful for providing therapeutic massage.

It was only after extensive experimentation that the present successful composition was created. After many trials, and experiments, the presently disclosed composition was discovered that functions as a candle, but also produces a liquefiable massage oil from the melting candle. It is a hallmark of chemistry that when composition is at an equilibrium between solid and liquid states, the temperature of the liquid-solid equilibrium is the melting temperature of the solid form. Thus, the new composition provides a massage oil, that when warmed sufficiently to melt a portion of the composition, the temperature of the liquid will not rise so high as to become undesirable for its purpose, application to the skin of a patient. This desirable characteristic is neither taught nor suggested elsewhere by any skilled artisan.

Melted paraffin wax has also been used for the application to skin for the purpose of removing unwanted hair or for exfoliating skin surfaces. A disadvantage of such wax applications, is that paraffin waxes have a relatively high melting temperature, and when applied to bare human skin at that temperature can cause discomfort and or scalding, and possibly injury.

An extensive series of experiments were conducted to attempt to produce a composition useful for providing a warm massage oil that could be safely applied directly to the skin, particularly human skin that is to be massaged. One of the several obstacles that was necessary to overcome was the difficulty in producing a candle that remains solid at ambient room temperatures (i.e. 20° C.), yet melts at a temperature that is tolerable to the bare human skin (i.e. about 50° C.), and generally remains liquid at about body temperature. Moreover, a number of compositions were attempted that did not remain homogenous when being mixed, and tended to separate into phases after cooling. As the following disclosure describes, one embodiment of the present disclosure is a composition that could be functionally melted by a burning candle wick, and deliver a massage oil composition that can be applied directly to the skin, and provide beneficial substances for use during massage therapy. Moreover, the ingredients used in the embodied composition are preferably hypoallergenic, such that allergic reaction in users is minimized. While a variety of potential ingredients for making a soy oil candle are known, extensive experimentation was necessary in order to produce a soy based candle that provided the therapeutic benefits sought. Through trial and error, the present embodiments were discovered.

For instance, as utilized herein, in comparison to Brown, U.S. Pat. No. 4,360,387, when less than 25% jojoba oil is used, a grainy, low strength, slushy appearance occurs in the wax, while the presently disclosed composition utilizes only up to 20% jojoba oil. Applicant thus utilizes a concentration of jojoba oil that is not desired according to the teachings of Brown. This highlights the extensive array of variables that confront the artisan in achieving the functional method claimed, even if the desired result were known. It is clear that the Applicant's claimed invention is much more than merely a candle. The Applicant's invention provides a candle that utilizes a heat source (such as burning wick) to liquefy a massage oil composition, that when melted is tolerable to the touch and this composition provides the massage oil that is used with the claimed method.

Initial efforts at producing the embodied composition utilized a soybean based wax, Soy 415 (ADM, Minneapolis, Minn.). Many attempts were made to combine Soy 415 with other beneficial ingredients, yet, were unsuccessful, as the resulting melted liquid was too viscous, or heavy, and remained on the surface of skin to which it was applied, much in the manner of melted paraffin. While it was desirable that the melted composition, after liquification, would be absorbed into the skin surface after massage, Soy 415 based compositions were not readily absorbed into the skin, thus depositing a heavy, undesirable, residue. Additional similar soy products from the manufacturers Bittercreek and Cargill were also tested, but produced similar undesirable results.

In an attempt to produce a composition that was liquefiable at near human body temperature (i.e., a melting point between 37° and 50° C.) that could be combined with other beneficial ingredients, compositions incorporating a partially hydrogentated soy oil were tested. An additional advantage of such oils is that they are generally recognized as safe for human consumption, even if not readily edible as food. Thus while the present embodiments are not meant to be consumed as food, there is a low risk of harm if accidentally ingested. Not all ingredients that might be incorporated in a liquefiable massage oil composition will unfortunately blend readily with soy and not create black soot when in contact with a burning candle wick. From the wide panoply of available butters, waxes and oils, certain of those which would blend readily with soy, that would provide therapeutic benefits to the skin and also not be harmful if accidentally eaten, experimentation demonstrated that the claimed embodiments would achieve the desired goals.

Extensive study of the practices of the health spa industry for producing desirable qualities in skin demonstrated that jojoba oil, shea butter, and aloe, of the many skin care products available, had many of the desired qualities.

Jojoba oil is desirable for a number of reasons. Jojoba oil with an Oxidative Stability Index of approximately 60, is relatively more shelf-stable when compared with other vegetable oils, such as safflower oil, canola oil, and almond oil. It as also believed that the oxidative stability of jojoba oil could also provide an increased oxidative stability (and shelf life) to compositions into which it was included. Jojoba oil, unlike common vegetable oils, is more stable in candles than petroleum based mixtures, and also is advantageously beneficial compared to paraffin oils when applied to the skin. Jojoba oil is chemically very similar to human serum. Jojoba oil is sometimes included as an ingredient in cosmetics and personal care products, such as skin care and hair care products. Jojoba oil possesses a fungicidal properties, and can be used for controlling mildew. Jojoba oil is also edible. It was thus highly desirable to incorporate jojoba oil as a necessary part of the liquifiable massage oil composition.

Shea butter is desirable as an ingredient of the liquefiable massage oil composition especially for its cosmetic properties as a moisturizer and emollient. Shea butter is also a known anti-inflammatory agent, and is marketed as being effective at treating the following conditions: fading scars, eczema, burns, rashes, acne, severely dry skin, blemishes, dark spots, skin discolorations, chapped lips, stretch marks, wrinkles, in lessening the irritation of psoriasis, and for providing ultraviolet sun light protection. When applied to the skin, Shea butter absorbs rapidly without leaving a greasy feeling and also for providing a skin softening effect. Though not incorporated into the liquefiable massage oil composition for that purpose, Shea butter is also edible.

Aloe is desirable as an ingredient of the liquefiable massage oil composition especially for its presumed regenerative properties, particularly as a healing moisturizer. The plant Aloe vera has been used topically for thousands of years to treat wounds, skin infections, burns, and numerous other dermatologic conditions. Traditionally, aloe extracts, particularly the plant juices, have been used as a moisturizer, and studies suggest aloe extracts may effectively reduce skin dryness. Though aloe extracts were a most desirable ingredient, incorporation into a massage oil candle was difficult, because extracts are typically water based and water-soluble and thus do not mix readily with non-polar ingredients.

Initial attempts to incorporate jojoba oil in a soy based candle composition were unsuccessful, because over a certain percentage of jojoba oil, partially hydrogenated soy oil would not properly mix, and instead of solidifying into a uniform mass, the composition would separate into disparate phases. The biphasic composition was not desirable, and would not function as a liquefiable massage oil composition.

Experimentation demonstrated that Shea butter could be successfully incorporated into the liquefiable massage oil composition. It was determined that Shea butter in small proportions functioned as envisioned, but if the combined content of shea butter, jojoba oil and aloe extract was greater than 7%, the preparation failed to have desired properties of homogeneity, separating into phases, and prone to seepage of oils from the candle body. If more than about 5% combined content of shea butter, jojoba oil and aloe extract was utilized, the resulting candle was prone to sputtering, and producing a sooty residue, both undesired qualities. If greater than about 20% jojoba oil was included, the candle body composition would curdle and have undesirable performance qualities. If greater than 10% shea butter is included, in addition to the previously disclosed characteristics, the candle fuel had poor burning qualities, was sooty and prone to scorching. Moreover, a composition with greater than 10% shea butter had undesirable emollient features, producing a greasy residue on the skin.

Attempts to incorporate aloe extracts into the liquefiable massage oil composition were unsuccessful because the various ingredients were immiscible with water based aloe products. Experimentation demonstrated that dehydrated aloe could be initially mixed with jojoba oil and shea butter, and then added to a small amount of melted soy flakes, thoroughly mixing all these ingredients, prior to adding this mixture into the remaining quantity of melted soy flakes. Utilization of dehydrated aloe also provided a benefit to the composition, in that water based aloe extracts, when included into a candle, would result in undesirable burning properties of the candle, namely sputtering of the wick.

To overcome the issues with undesired separation it was determined that the potential ingredients jojoba oil, shea butter and dehydrated aloe first needed to be mixed with a quantity of melted soy flakes. Once this mixture is fully blended, it can then be mixed with the remaining quantity of melted soy flakes. Specifically, for a 1 kg final quantity of the liquifiable massage oil composition, 10 g of jojoba oil, 10 g of shea butter, and 1 g of dehydrated aloe are slowly mixed into approximately 99 g of melted soy flakes maintained at about 190° F. While maintaining the temperature, once this mixture is thoroughly blended, it is then mixed with approximately 880 g of melted soy flakes. While it is desired the jojoba oil be included in the final composition, typical percentage compositions of the liquefiable massage oil composition include, from about 0.5 to 5% jojoba oil; from about 0.5 to 2% shea; butter; no more than 1/100% dehydrated aloe (if aloe is to be included) with the remainder be composed of soyflakes, from about 90 to 99%. Following thorough mixing, the composition can be poured into a suitable container, the composition turning into a solid when allowed to cool. When the composition is heated either with a burning wick or an other type of candle warmer, such as a resistive electric heater, i.e. and Ohmic heater, the candle fuel composition liquifies into a warm massage oil of uniform composition. The composition thus functions both as a desirable new alternative massage oil and as clean burning, residue free candle.

Considering the qualities herein disclosed the total combined content of the emollients shea butter, jojoba oil and aloe is preferred to be between about 2 percent by weight, and about 5 percent by weight. Lesser quantities of these emollients lack full therapeutic utility, while greater combined amounts either present manufacturing problems, or poor burning and stability problems. A preferred embodiment utilizes Soy 402 flakes available from ADM. Further preferred compositions in a composition of from about 93 to about 98 weight percent soy flakes, from about 0.005 to about 0.7 weight percent shea butter, and from about 1 to about 6 weight percent jojoba oil. A further preferred embodiment is a composition comprising the candle apparatus wherein said the candle fuel comprises from about 96 to about 98 weight percent soy flakes, from about 0.005 to about 0.7 weight percent shea butter, and from about 1 to about 2 weight percent jojoba oil. In yet another embodiment, said candle fuel comprises about 98 weight percent Soy 402 flakes, about 0.05 weight percent shea butter, and about 2 weight percent jojoba oil. Furthermore, aloe extract may be added to 0.5%, preferably 0.01 percent.

After producing a uniform mixture, other desirable ingredients, such as fragrance and color agents can be added, thereby reducing the relative quantities of the other ingredients.

A number of emollients are envisioned for inclusion in the candle apparatus for producing a liquefiable massage oil composition. Such emollients include, shea butter, jojoba oil, lanolin, and aloe extracts. Furthermore, if desired, Fragrance and or dye may be included in the candle fuel forming the candle body. For instance, from about 1 to about 5 weight percent fragrance; and about 1 weight percent dye may be included by slightly reducing the amount of soy flakes or the total volume of the final composition.

An exemplary process for constructing a massage oil contains the following steps. Soy flakes are melted to about 190° F. and blended with melted shea butter having approximately one sixteenth the weight of the soy oil, and blended to uniformity. Coloring dye is added if desired. The resulting mixture is cooled to between 110° F. and 130° F., and more preferably to about 120° F., whereupon desired carrier oils (e.g., jojoba oil, dehydrated aloe mixed with heated soy oil) are added. Desired fragrance (such as for aromatherapy), other essential oils, and any other therapeutic ingredients are also added.

After the resulting mixture cools to a temperature between 95° F. and 110° F. (and more preferably about 100° F.), it is poured into a candle container/housing for hardening/solidification.

An exemplary mixture (with the range of the percent of the total weight) of ingredients for a massage oil candle according to the present invention is as follows: pure soy flakes 10-85; shea butter 0.5-10; dehydrated aloe and heated soy oil combined 0-20; jojoba oil 0-20; fragrance 0-5; and dye 0-1.

According to the present invention, the skin care agent used in the candle fuel mixture need not be restricted to massage oil. Rather, the skin care agent can be a soap agent. That is, as the solid candle fuel mixture melts, it is converted to a non-toxic liquefied soap which may be safely used on the human skin. While the candle fuel mixture in a “soap candle” may contain similar carrier oils found in “Massage oil candles,” an additional ingredient of the soap candle fuel mixture is glycerin.

An exemplary process for manufacturing a soap candle according to the present invention uses the following steps. Soy flakes are melted and maintained between 150° F. and 190° F., and more preferably between 175° F. and 185° F. Glycerin (in and amount ranging from half the weight of soy, to two and one-half times the weight of soy) is heated to a temperature between 150° F. and 170° F., without scorching it. The soy oil and glycerin are blended to uniformity. Shea butter in an amount ranging front one sixteenth to one-eighth of the weight of soy flakes is added to the mixture. Prior to final cooling, desired carrier oils, fragrances and dyes are added to the mixture. The mixture is then cooled until it solidifies.

An exemplary mixture (with the range of the percent of the total weight) of ingredients for a soap candle according to the present invention is as follows: pure soy flakes 10-85; glycerin 25-85; jojoba oil 1-25; dehydrated aloe 1-5; shea butter 0.5-10. Fragrance may be added in an amount to 5% fragrance a coloring dye may be added to about 1%, if desired.

In the case of either the massage oil candle or the soap candle, a wick is placed in the candle fuel mixture prior to cooling if a “wicked” version is desired.

As a business option, the massage oil candle and the soap candle of the present invention may be sold in a convenient package to consumers, A consumer can used liquid soap from a melted soap candle to cleanse his or her skin, and then apply massage oil or other emollients formed from a melted massage oil candle.

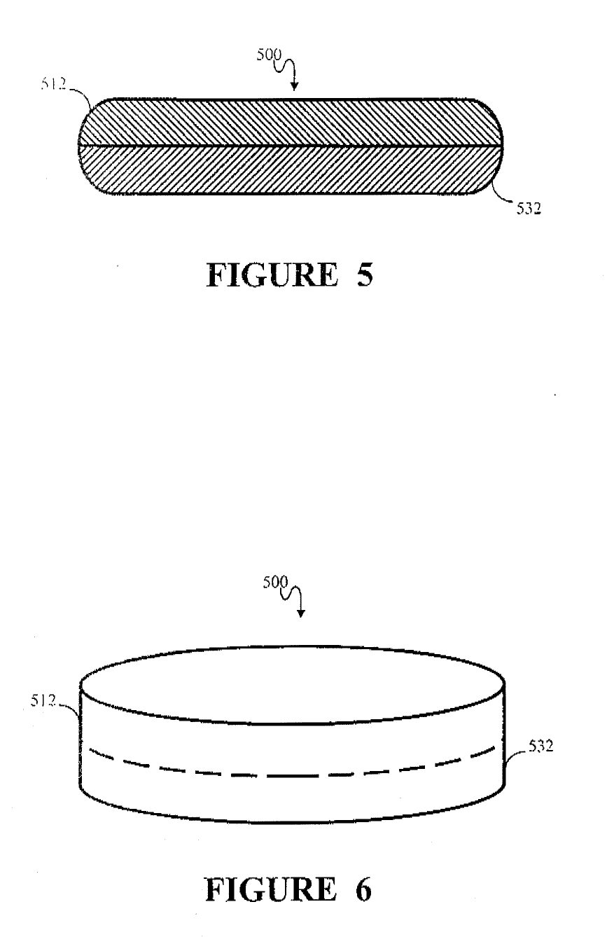

In addition to producing candle apparatuses, the present invention also applies to novel soap articles of manufacture such as the one numbered 500 in FIG. 5. According to a present-inventive manufacturing process, a soap article is produced with two distinct layers in which one layer 512 is primarily directed to cleansing and softening the skin, and another layer 532 is primarily directed to exfoliating and moisturizing the skin. An isometric view of the soap article 500 is illustrated in FIG. 6.

The layered soap article 500 of the present invention is produced according to the following exemplary steps. Soy flakes are melted at a temperature between 160° F. and 200° F. Next, glycerin and a carrier oil such as jojoba oil, shea butter, or aloe oil, and vitamin E are added to the melted soy oil. The mixture is blended to uniformity at temperatures between 140° F. and 200° F., and preferably between 140° F. and 180° F., and more preferably between 160° F. and 180° F. Colorants and fragrances are then added to the mixture, and then blended to uniformity. The resulting mixture is then poured in a mold, whereupon two separate layers form by spontaneous separation upon cooling.

An exemplary mixture (with the range of the percent of the total weight) of ingredients for an exfoliating and moisturizing soap article is as follows; soy flakes 5-30; glycerin 25-85; shea butter 0.5-10; jojoba oil 0.5-20; dehydrate aloe 0.5-20(if added); vitamin E 0-2 (if added); and an exfoliating ingredient 0-1 (if added);

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

It is expected that there could be numerous variations of the design of this invention. For instance, the composition will also work as a lip balm, lipstick and a solid perfume, a semi-solid balm for scars, and a massage oil that is effective treatment of psoriasis.

This product was created as massage oil with therapeutic qualities that can also burn as a candle

Finally, it is envisioned that the components of the device may be constructed of a variety of materials.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. All references are hereby incorporated by reference.

I claim:

1. A candle apparatus comprising:
a) a candle housing;
b) a candle body deposited in said candle housing, said candle body comprising a solidified liquefiable homogenous composition including
a flammable candle fuel primarily derived from soy that burns evenly without appreciable production of soot or discoloration,
two or more emollients suitable for application to skin, said emollients comprising shea butter, jojoba oil, lanolin, and aloe extracts;
c) a candle liquefier;
wherein liquefying said candle body using the candle liquifier into liquefied therapeutic oil for human skin, said liquefied therapeutic oil being tolerable to the touch, said liquefied therapeutic oil remaining liquid at temperatures greater than about 35° C.

2. The candle apparatus of claim 1, wherein said candle fuel comprises
from about 93 to about 98 weight percent soy flakes,
from about 0.005 to about 0.7 weight percent shea butter, and
from about 1 to about 6 weight percent jojoba oil.

3. The candle apparatus of claim 2, wherein said candle fuel comprises
from about 96 to about 98 weight percent soy flakes,
from about 0.005 to about 0.7 weight percent shea butter, and
from about 1 to about 2 weight percent jojoba oil.

4. The candle apparatus of claim 3, wherein said candle fuel comprises
about 98 weight percent Soy 402 flakes,
about 0.05 weight percent shea butter, and
about 2 weight percent jojoba oil.

5. The apparatus of claim 1, wherein said candle fuel liquefier comprises a wick for burning the candle body.

6. The apparatus of claim 1, wherein said heating source is an electric resistive heater.

7. The apparatus of claim 1, wherein said candle housing further comprises a reservoir for collecting liquefied oil.

8. The apparatus of claim 1 wherein said emollient includes aloe.

9. The apparatus of claim 1, further comprising:
from about 1 to about 5 weight percent fragrance; and
about 1 weight percent dye.

10. A method of therapeutically treating skin comprising:
a) providing a candle comprising:
1) a candle housing;
2) a candle body deposited in said candle housing, said candle body comprising a solidified liquefiable homogenous composition including a flammable candle fuel primarily derived from soy that burns evenly without appreciable production of soot or discoloration,
two or more emollients suitable for application to skin, said emollients comprising shea butter, jojoba oil, lanolin, and aloe extracts
3) a candle liquefier comprising a wick;
b) liquefying said candle body by igniting the candle liquifier into liquefied therapeutic oil for human skin, said liquefied therapeutic oil being tolerable to the touch, said liquefied therapeutic oil remaining liquid at temperatures greater than about 35° C.
c) applying to the skin, liquefied therapeutic oil produced.
